(19) United States
(12) Patent Application Publication
(10) Pub. No.: US 2025/0018203 A1
(43) Pub. Date: Jan. 16, 2025

(54) ENDOVASCULAR NERVE TISSUE STIMULATION THERAPY

(71) Applicant: Covidien LP, Mansfield, MA (US)

(72) Inventors: Varun Umesh Kashyap, Palo Alto, CA (US); Mark P. Ashby, Laguna Niguel, CA (US); Scott R. Stanslaski, Shoreview, MN (US); Alexander A. Khalessi, Rancho Santa Fe, CA (US); Kristin N. Hageman, Dayton, MN (US); Duncan Z. Ashby, Riverside, CA (US); Kevin V. Nguyen, Westminster, CA (US); Ashok R. Nageswaran, Irvine, CA (US); Bernard Q. Li, Plymouth, MN (US); Yao-Chuan Chang, New Brighton, MN (US); Kanthaiah Koka, Valencia, CA (US)

(21) Appl. No.: 18/770,452

(22) Filed: Jul. 11, 2024

Related U.S. Application Data
(60) Provisional application No. 63/513,688, filed on Jul. 14, 2023.

(51) Int. Cl. A61N 1/375; A61F 2/00; A61F 2/95; A61M 25/00; A61M 25/01; A61N 1/05; A61N 1/36

(52) U.S. Cl. CPC A61N 1/37516; A61F 2/95; A61M 25/0082; A61N 1/0551; A61N 1/36135; A61F 2002/0081; A61M 2025/0166

(57) ABSTRACT

An example system includes processing circuitry; and an endovascular device includes an expandable substrate configured to be delivered endovascularly to a trial therapy delivery location in a patient; one or more first electrodes positioned on the expandable substrate, wherein the one or more first electrodes are configured to deliver nerve tissue stimulation trial therapy to a target nerve tissue of the patient; and one or more second electrodes positioned on the expandable substrate, wherein the one or more second electrodes are configured to sense one or more activation signals of the target nerve tissue, wherein the processing circuitry is configured to determine whether activation of the target nerve tissue at the trial therapy delivery location satisfies a target nerve tissue activation threshold based on the one or more sensed activation signals.

### CROSS-REFERENCE TO RELATED APPLICATIONS

This application claims the benefit of and priority to U.S. Provisional Application No. 63/513,688 filed Jul. 14, 2023, the entire disclosure of which is incorporated by reference herein.

### TECHNICAL FIELD

The disclosure relates to devices and techniques for performing neurostimulation therapy to determine a target nerve tissue location to deliver nerve tissue stimulation therapy.

### BACKGROUND

Neuromodulation by electrical stimulation of nerves, such as the vagus nerve, renal nerve, splenic nerve, etc., has been shown to be useful for a wide range of purposes. Some proposed systems for vagus nerve stimulation include cuff electrodes. Implantation of cuff electrodes for vagus nerve stimulation typically involves a relatively invasive and complex surgical procedure, as well as negative tissue interactions. Consequently, some proposed systems for vagus nerve stimulation include vascular access leads.

### SUMMARY

There is significant variation from patient to patient in response to nerve tissue stimulation therapy, such as amount of responsiveness to nerve tissue stimulation and/or locations of nerve tissue stimulation therapy in a particular patient that generate clinically desired responsiveness in that particular patient. The more invasive implantation procedures associated with nerve stimulation via cuff electrodes preclude testing responsiveness to nerve stimulation prior to finalizing positioning of the electrodes and engaging the cuff electrode to the nerve. Further, once installed on the nerve, there are negative long term tissue interactions associated with nerve stimulation via cuff electrodes. In general, the disclosure is directed to devices, systems, and techniques for performing trial nerve tissue stimulation of a patient via a device having an expandable substrate endovascularly delivered to a position near the target nerve tissue. In some examples, the disclosure is directed to devices, systems, and techniques for permanent implantation of a device for performing target nerve tissue stimulation, without the negative long term tissue interactions associated with nerve stimulation via cuff electrodes.

For example, a system may include processing circuitry and an endovascular device, the endovascular device comprising an expandable substrate, one or more first electrodes positioned on the expandable substrate, and one or more second electrodes positioned on the expandable substrate. The one or more first electrodes are configured to deliver nerve tissue stimulation trial therapy to a target nerve tissue of the patient. The one or more second electrodes are configured to sense one or more activation signals of the target nerve tissue. In some examples, the processing circuitry may be configured to receive the one or more activation signals in response to delivery of the nerve tissue stimulation trial therapy to the target nerve tissue via the one or more first electrodes at a trial therapy delivery location. The processing circuitry may then determine whether activation of the target nerve tissue at the trial therapy delivery location satisfies a target nerve tissue activation threshold based on the one or more sensed activation signals.

In response to determining that the activation of the target nerve tissue at the trial therapy delivery location satisfies the target nerve tissue activation threshold based on the one or more sensed activation signals, the processing circuitry may generate a first indication that the trial therapy delivery location of at least one of the first electrodes is acceptable to deliver nerve tissue stimulation therapy.

In response to determining that the activation of the target nerve tissue at the trial therapy delivery location does not satisfy the target nerve tissue activation threshold based on the one or more sensed activation signals, the processing circuitry generate a second indication that the trial therapy delivery location of the first electrodes is not acceptable to deliver nerve tissue stimulation therapy.

The systems, devices, and methods described herein for trialing nerve tissue stimulation therapy may help determine adequate and/or optimum location for placement of electrodes to deliver nerve tissue stimulation therapy and provide assistance for placement of electrodes of a nerve tissue stimulation device at the adequate and/or optimum therapy delivery location in a particular patient. The systems, devices, and methods described herein for trialing nerve tissue stimulation therapy may help determine adequate and/or optimum stimulation configuration and/or stimulation parameter selection. In some examples, the systems, devices, and methods described here herein may help provide optimum stimulation configurations for a particular therapy based on sensing and activation of selected neural fibers of nerve tissue. In some examples, the systems, devices, and methods described herein help provide implantation of nerve tissue stimulation device(s) at personalized patient locations. In some examples, the systems, devices, and methods described herein provide more accurate, more efficient, and/or less invasive implantation of a device or system to deliver nerve tissue stimulation therapy, which lead to improved patient responsiveness and reduced negative long term tissue interactions from nerve tissue stimulation therapy.

In one example, the disclosure describes a system comprising processing circuitry; and an endovascular device comprising an expandable substrate configured to be delivered endovascularly to a trial therapy delivery location in a patient; one or more first electrodes positioned on the expandable substrate, wherein the one or more first electrodes are configured to deliver nerve tissue stimulation trial therapy to a target nerve tissue of the patient; and one or more second electrodes positioned on the expandable substrate, wherein the one or more second electrodes are configured to sense one or more activation signals of the target nerve tissue, wherein the processing circuitry is configured to: in response to delivery of the nerve tissue stimulation trial therapy to the target nerve tissue via the one or more first electrodes at the trial therapy delivery location, receive, via the one or more second electrodes, the one or more sensed activation signals; determine whether activation of the target nerve tissue at the trial therapy delivery location satisfies a target nerve tissue activation threshold based on the one or more sensed activation signals; in response to determining that the activation of the target nerve tissue at the trial therapy delivery location satisfies the target nerve tissue activation threshold based on the one or more sensed activation signals, generate a first indication that the trial therapy delivery location of at least one of the first electrodes is acceptable to deliver nerve tissue stimulation therapy; and in response to determining that the activation of the target nerve tissue at the trial therapy delivery location does not satisfy the target nerve tissue activation threshold based on the one or more sensed activation signals, generate a second indication that the trial therapy delivery location of the first electrodes is not acceptable to deliver nerve tissue stimulation therapy.

In another example, this disclosure describes an endovascular device comprising a stent substrate configured to be delivered endovascularly to a trial therapy delivery location in a patient; a plurality of fingers protruding from a plurality of positions on the stent substrate; one or more indicators; and a plurality of electrodes, the plurality of electrodes are configured to perform one or more of: deliver nerve tissue stimulation trial therapy to a target nerve tissue of the patient, and sense one or more activation signals of the target nerve tissue, wherein one of the plurality of electrodes or one of the one or more indicators are mounted on a respective finger of the plurality of fingers.

In another example, this disclosure describes an endovascular device comprising a stent substrate including a plurality of struts and configured to be delivered endovascularly to a trial therapy delivery location in a patient; and a plurality of electrodes, the plurality of electrodes are configured to perform one or more of: deliver nerve tissue stimulation trial therapy to a target nerve tissue of the patient, and sense one or more activation signals of the target nerve tissue, wherein one of more of the plurality of electrodes are wrapped around a respective single strut of the plurality of struts and attached to an independent and insulated lead wire.

The details of one or more examples are set forth in the accompanying drawings and the description below. Other features, objects, and advantages of the disclosure will be apparent from the description and drawings, and from the claims.

The above summary is not intended to describe each illustrated example or every implementation of the present disclosure.

### BRIEF DESCRIPTION OF DRAWINGS

FIG. 1 depicts a conceptual diagram of an example neurostimulation system in accordance with one or more techniques of the present disclosure.

FIGS. 2A-2D depict conceptual diagrams of examples of an endovascular device, in accordance with one or more techniques disclosed herein.

FIG. 3 depicts a schematic diagram of an example of a medical device in accordance with one or more techniques of the present disclosure.

FIG. 4 is a flow diagram illustrating example techniques that may be performed by a one or more of a system or device, in accordance with one or more techniques disclosed herein.

### DETAILED DESCRIPTION

Traditional implantable vagal nerve stimulation (VNS) devices may require the surgical installation of nerve cuffs directly onto the vagus nerve. This is a highly invasive procedure that is performed by a neurosurgeon and lead wires are tunneled subcutaneously to an implanted neurostimulation device. In addition to being highly invasive, these VNS devices suffer performance degradation with time as the effects of scaring and gliosis increase impedance and raise stimulation thresholds. Further, because of the invasiveness of these procedures, therapeutic trialing, therapeutic confirmation, and acute therapies are not feasible for these procedures.

In some examples, VNS may assist in treatment for epilepsy. For use in epilepsy VNS is approved as therapy for focal (partial) seizures in adults and children four years of age and older when medications haven't controlled their seizures. For use in depression VNS is approved for adults 18 years of age or older who have long-term or recurrent (repeating) major depression that hasn't responded to four or more antidepressant treatments.

Additionally, in some examples, VNS may assist in stroke by limiting ischemia reperfusion injury. After a myocardial infarct or stroke, reperfusion therapies (surgery or drugs) are given to restore blood flow. However, due to the restoration of blood, flow induced local damage occurs, which is called ischemia reperfusion injury. This will induce local accumulations of chemical mediators such as reactive oxygen species (ROS) production, inflammatory cytokines, bradykinin, etc. Thus, the inflammatory state is worsened. The inflammatory compounds will trigger sensory signaling, which might lead to a reduced organ vagus activity and sympathetic overdrive. VNS may treat reperfusion damage as the inflammatory state may be lowered by increasing parasympathetic drive. VNS is additionally a promising therapy for a wide array of other medical issues.

In general, the disclosure is directed to devices, systems, and techniques for performing trial nerve tissue stimulation on a patient to determine adequate and/or optimum placement of electrodes to deliver nerve tissue stimulation therapy. For example, a system may include processing circuitry and an endovascular device, the endovascular device comprising an expandable substrate, one or more first electrodes positioned on the expandable substrate, and one or more second electrodes positioned on the expandable substrate. The one or more first electrodes are configured to deliver nerve tissue stimulation trial therapy to a target nerve tissue of the patient. The one or more second electrodes are configured to sense one or more activation signals of the target nerve tissue. In some examples, the processing circuitry may be configured to receive the one or more activation signals in response to delivery of the nerve tissue stimulation trial therapy to the target nerve tissue via the one or more first electrodes at the trial therapy delivery location. The processing circuitry may then determine whether activation of the target nerve tissue at the trial therapy delivery location satisfies a target nerve tissue activation threshold based on the one or more sensed activation signals.

In response to determining that the activation of the target nerve tissue at the trial therapy delivery location satisfies the target nerve tissue activation threshold based on the one or more sensed activation signals, the processing circuitry may generate a first indication that the trial therapy delivery location of at least one of the first electrodes is acceptable to deliver nerve tissue stimulation therapy.

In response to determining that the activation of the target nerve tissue at the trial therapy delivery location does not satisfy the target nerve tissue activation threshold based on the one or more sensed activation signals, the processing circuitry may generate a second indication that the trial therapy delivery location of the first electrodes is not acceptable to deliver nerve tissue stimulation therapy.

The techniques of this disclosure may trial nerve tissue stimulation therapy on patients to test therapy responsiveness and determine adequate and/or optimum placement of electrodes in a lumen of a patient configured to deliver nerve tissue stimulation therapy. In some examples, therapy responsiveness is determined either by the endovascular device itself or by another implantable or wearable nerve tissue stimulation therapy delivery device. The systems, devices, and/or methods described herein may provide more accurate, more efficient, and/or less invasive implantation of a device or system to deliver nerve tissue stimulation therapy, which may lead to improved patient responsiveness from nerve tissue stimulation therapy.

A device or system configured to provide nerve tissue stimulation trial therapy, as described in examples above and below, would be useful for treating a variety of illnesses including, but not limited to: seizure, ictal tachycardia, stroke, heart failure (HF), congestive HF, cardiac arrhythmia, hypertension, reperfusion damage, cardiac ischemia, brain ischemia, traumatic brain injury, surgical or non-surgical acute kidney injury; inability of the intestine (bowel) to contract normally and move waste out of the body; postoperative ileus; postoperative cognitive decline or postoperative delirium; asthma; sepsis; bleeding control; myocardial infarction reduction; and dysmotility and obesity. Treating any of these diseases may improve patient outcomes by shortening length of hospital stays and reducing medical costs.

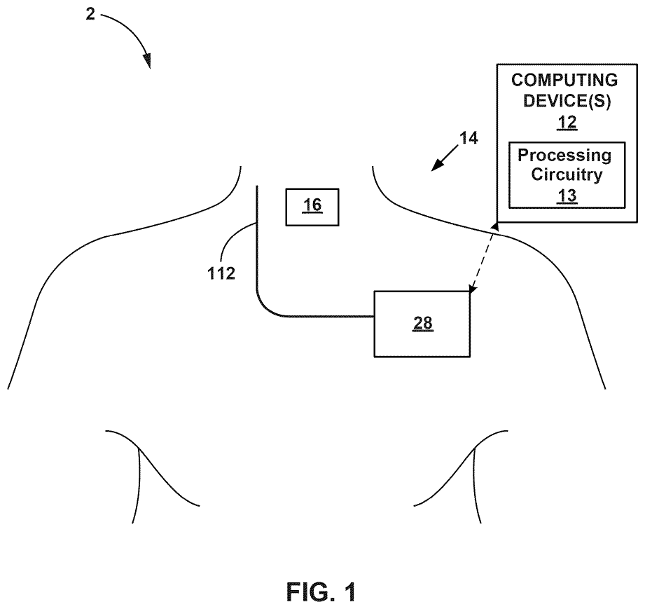

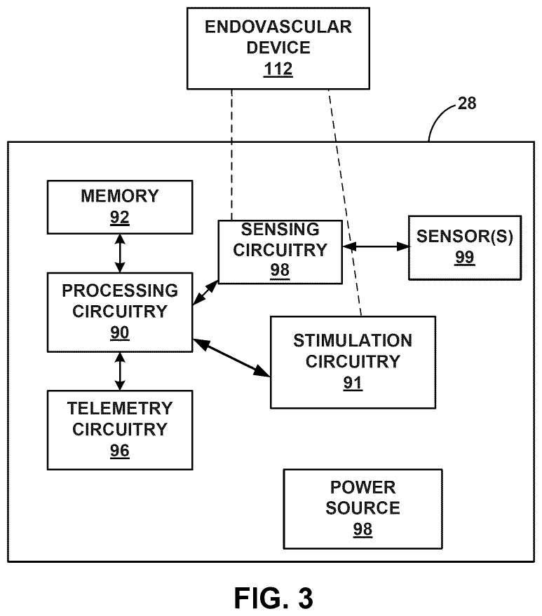

FIG. 1 is a conceptual diagram illustrating an example neurostimulation trial system 2. Neurostimulation trial system 2 includes processing circuitry and an endovascular device 112. In some examples, the processing circuitry may include processing circuitry 90 (shown in FIG. 3) positioned in medical device (MD) 28. In some examples, the processing circuitry may include processing circuitry 13 positioned in one or more computing devices 12. In some examples, processing circuitry 13, 90 may be positioned in one or more of an MD 28 or computing device(s) 12. In some examples, processing circuitry may be positioned in one or more of an MD 28 or computing device(s) 12 and/or second device 16.

In some examples, MD 28 may be an implantable medical device (IMD). In other examples, MD 28 may be outside the patient 14. Endovascular device 112 may include an expandable substrate 120 (not shown in FIG. 1) configured to be delivered endovascularly to a trial therapy delivery location in patient 14. Endovascular device 112 may include one or more first electrodes (not shown in FIG. 1) positioned on the expandable substrate and configured to deliver nerve tissue stimulation trial therapy, such as delivering stimulation signal(s), to a target nerve tissue of patient 14. Endovascular device 112 may include one or more second electrodes (not shown in FIG. 1) positioned on the expandable substrate and configured to sense or more activation signals of the target nerve tissue. In some examples, neurostimulation trial system 2 may deliver nerve tissue stimulation trial therapy, for example one or more stimulation pulses, to a target nerve tissue via one or more first electrodes, the one or more second electrodes may sense one or more activation signals of the target nerve tissue in response to the delivery of the nerve tissue stimulation trial therapy to the target nerve tissue, and the processing circuitry may receive the one of more activation signals sensed by the one or more second electrodes.

In some examples, nerve tissue may include, but not be limited to, one or more of nerve tissue of a vagus nerve, splenic nerve, renal nerve, sacral nerve, pudendal nerve, phrenic nerve, radial nerve, median nerve, ulnar nerve, splanchnic nerve, and/or peripheral nerve. In some examples, nerve tissue may include one or more of nerve tissue in a spinal cord and/or brain. In some examples, alternatively or additionally to being directed to nerve tissue, the systems, devices, and techniques described herein may be directed to other evoke-related tissue or organs other than nerve tissue.

Processing circuitry may then determine whether activation of the target nerve tissue at the trial therapy delivery location satisfies a target nerve tissue activation threshold based on the one or more sensed activation signals. In response to determining that the activation of the target nerve tissue at the trial therapy delivery location satisfies the target nerve tissue activation threshold based on the one or more sensed activation signals, processing circuitry may generate a first indication the trial therapy delivery location of at least one of the first electrodes is acceptable to deliver nerve tissue stimulation therapy. In some examples, a trial therapy delivery location is acceptable if the target nerve tissue is determined to be adequately stimulated to potentially obtain desired patient responsiveness.

In response to a determination that the activation of the target nerve tissue at the trial therapy delivery location does not satisfy the target nerve tissue activation threshold based on the one or more sensed activation signals, processing circuitry may generate a second indication that the trial therapy delivery location of the first electrodes is not acceptable. In some examples, the second indication may include an indication to adjust one or more trial therapy delivery parameters. In some examples, the trial therapy delivery parameters may include one or more of amplitude of the stimulation signal(s) of the nerve tissue stimulation trial therapy, pulse width of the stimulation signal(s) of the nerve tissue stimulation trial therapy, frequency of the stimulation signal(s) of the nerve tissue stimulation trial therapy, polarity of the stimulation signal(s) (e.g., monopolar, bipolar, tri-polar or multi-polar), electrode type of the one or more first electrodes, which electrodes of a set of possible first electrodes are used to deliver the neurostimulation therapy, or location of the one or more first electrodes used to deliver the therapy in a lumen of patient 14. In some examples, the location may include one or more of a longitudinal location on the longitudinal axis with respect to the lumen the endovascular device 112 is inside or a circumferential location with respect to the lumen the endovascular device 112 is inside.

Neurostimulation trial system 2 may communicate with a second device 16 that may include one or more sensors. In some examples, sensors could be positioned on one or more of endovascular device 112 or MD 28. In some examples, one or more sensors of second device 16, MD 28, and/or endovascular device 112 may be for sensing one or more of an electroencephalogram (EEG) signal, electrocardiogram (ECG) signal, respiration, blood pressure, activity level, electromyography (EMG) signal, evoked potential, evoked compound action potential (eCap), intrinsic nerve activity, and/or electrophysiology (EP), and may comprise electrodes, accelerometers, or any other known sensors. One or more sensors of second device 16 may additionally or alternatively include one or more sensors to identify changes in blood pressure, pulse, blood flow, respiration, temperature, activation of muscles, or any metrics indicative of nerve stimulation.

Computing device(s) 12 may be configured for wireless communication with MD 28 and/or second device 16. In addition, MD 28 may be configured for wireless communication with second device 16. Computing device(s) 12 may retrieve data from MD 28 that was collected and stored by the MD 28. In some examples, computing device(s) 12 may take the form of personal computing devices of patient 14. For example, computing device 12 may take the form of a smartphone of patient 14, and/or a smartwatch or other smart apparel of patient 14. In some examples, computing device(s) 12 may be any computing device configured for wireless communication with MD 28 such as a desktop, laptop, or tablet computer. In some examples, computing device(s) 12 may be a dedicated programming device for MD 28, e.g., a clinician programmer or a patient programmer. Computing device(s) 12 may communicate with MD 28 and/or second device 16 according to standards or protocols, such as 3G, 4G, 5G, WiFi (e.g., 802.11 or 802.15 ZigBee), Bluetooth®, or Bluetooth® Low Energy (BLE).

In some examples, the second device 16 may be an IMD and be configured to be implanted in patient 14. In some examples, the second device 16 may be configured to be implanted in a target site of the patient or disposed over the skin of the patient at a target site. In some examples, the second device 16 may be a wearable device. In some examples, the sensor device may be a relatively small device, and may be placed (e.g., inserted) under or over the skin at the back of the patient's neck or base of the skull. The second device 16 may detect one more physiological parameters of a patient (e.g., electrical activity corresponding to brain activity in particular regions of the patient's brain, ECG data, motion data, etc.). The second device 16 may be communicatively coupled to computing device(s) 12 and/or MD 28, for example via a wireless connection.

MD 28 may receive information from second device 16 directly or via computing device(s) 12. For example, MD 28 may communicate directly with computing device(s) 12 which communicates with second device 16. In some examples, MD 28 may communicate directly with second device 16 and receive information directly from second device 16. In some examples, second device 16, computing device(s) 12, and/or MD 28 may be communicatively coupled with each other over a network. In some examples, two or more of second device 16, computing device(s) 12, and MD 28 may be combined into a single physical device.

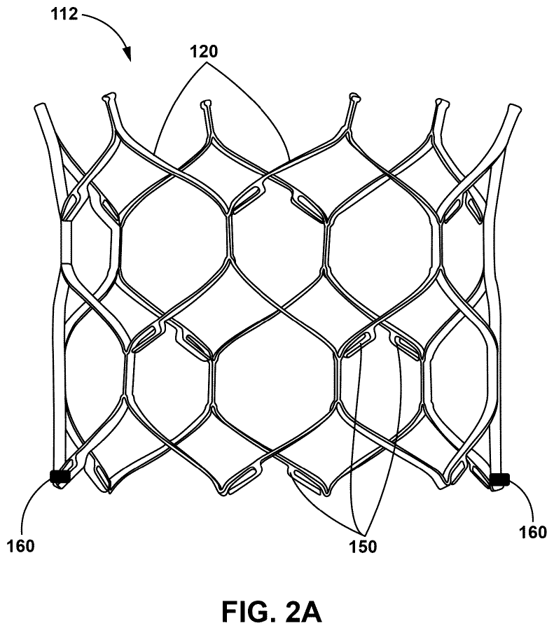

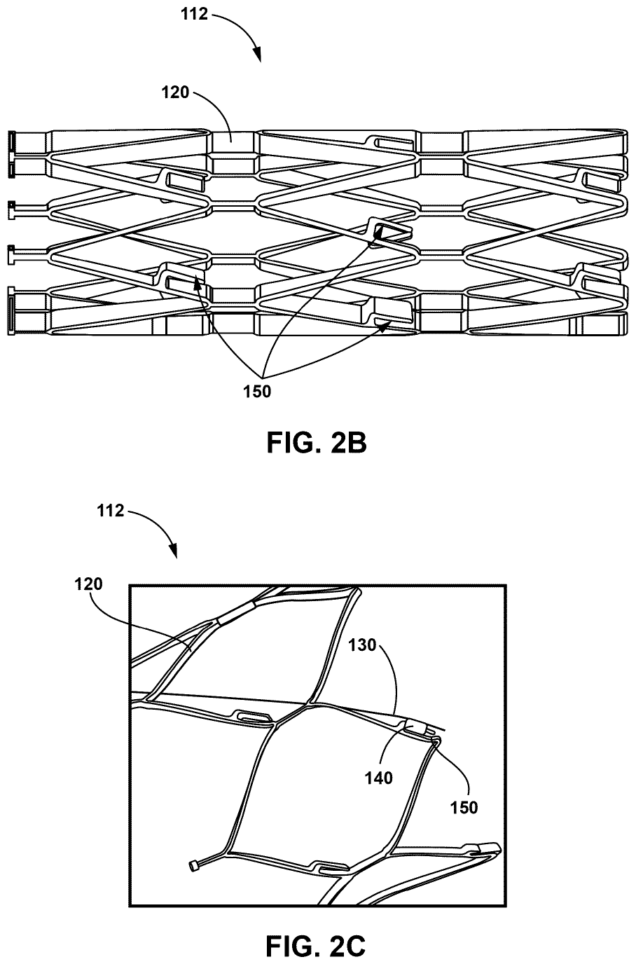

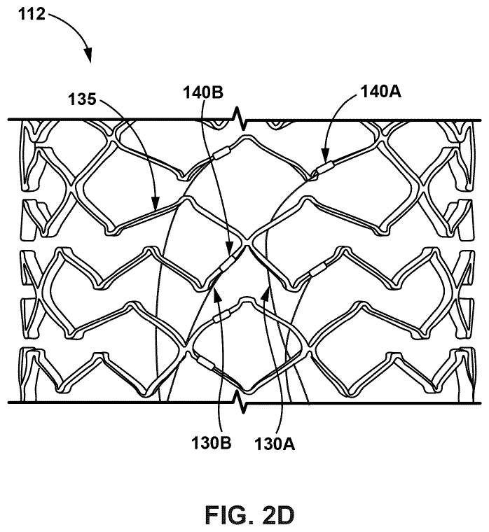

FIGS. 2A-2D are examples of conceptual diagrams of endovascular device 112. In some examples, endovascular device 112 may be comprised of biocompatible materials. Endovascular device 112 may include an expandable substrate 120, one or more first electrodes 140A positioned on the expandable substrate 120 and configured to deliver nerve tissue stimulation trial therapy to a target nerve tissue, and one or more second electrodes 140B positioned on the expandable substrate 120 and configured to sense one or more activation signals of the target nerve tissue. In some examples, first electrodes 140A and second electrodes 140B may collectively be referred to as a plurality of electrodes 140. Electrodes 140 may be referred to as stimulating electrodes, sensing electrodes, or stimulating/sensing electrodes. In some examples, electrodes 140 may be a stimulating and/or sensing electrode. For example, electrodes 140A may be stimulating electrodes, but, in some examples, may also be sensing electrodes. Electrodes 140B may be sensing electrodes, but, in some examples, may also be stimulating electrodes. In some examples, electrodes 140 may be configured to deliver stimulation nerve tissue stimulation trial therapy, such as by delivering stimulation signal(s) and/or sense activation signals such as by sensing electrical activity. In some examples, electrodes 140 may be one or more of ring electrodes or half ring electrodes. In some examples, electrodes 140 may be segmented electrodes. In some examples, electrodes 140 may be current steerable segmented electrodes. In some examples, the stimulating and/or sensing by one or more electrodes 140 may be monopolar, bipolar, tri-polar or multi-polar. In some examples, nerve tissue stimulation trial therapy may be delivered through spatially located electrodes to provide spatial steering and localized stimulation of target nerve tissue and/or target neural fibers.

In some examples, electrodes 140 may be positioned on a respective finger 150 of expandable substrate 120 with each electrode being attached to an independent and/or insulated lead wire 130 and extending from the electrode 140, which may preclude placing electrodes 140 at intersections of expandable substrate 120. In some examples, electrodes 140 may be a wrap-around electrode in which independent split electrodes are wrapped around an uninterrupted span of a single expandable substrate 120 strut 135, which may preclude placing electrodes 140 at intersections of expandable substrate 120. In some examples, each independent split electrode of a wrap-around electrode may have its own independent insulated lead wire 130 and extending from the electrode 140.

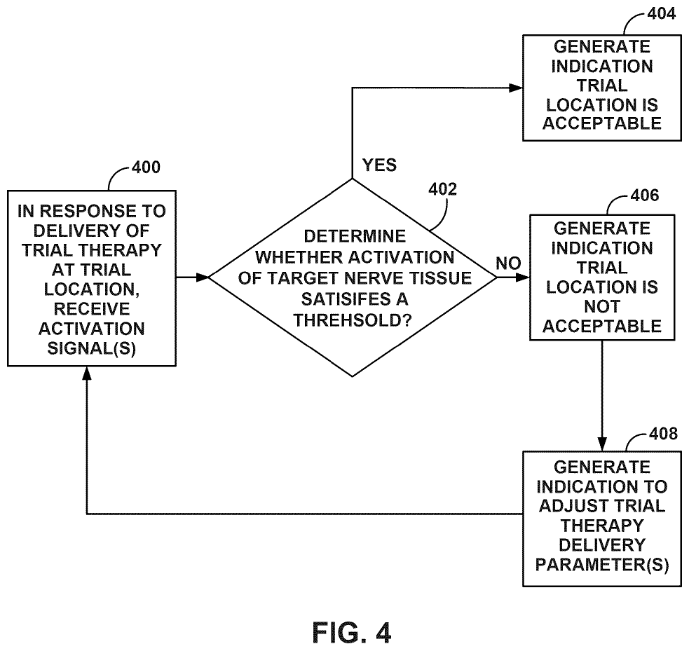

It is understood that any appropriate number of electrodes 140 may be provided. For example, expandable substrate 120 may comprise 16 electrodes or 32 electrodes. However, various other amounts of electrodes 140 may also be provided on expandable substrate 120. In some examples, each of the electrodes 140A may be electrically coupled to the stimulation circuitry 98 (FIG. 4) and may be controlled individually and/or in combination to provide stimulation signal(s) to one or more portions of target nerve tissue. The stimulation signal(s) through electrodes 140A may be provided in any appropriate manner, such as discussed further herein.

In some examples, one or more of electrodes 140B may be used to sense one or more physiological parameters of the patient. In some examples, the one or more sensed physiological parameters comprises one or more of an electroencephalogram (EEG) signal, electrocardiogram (ECG) signal, respiration, blood pressure, activity level, electromyography (EMG) signal, evoked potential, evoked compound action potential (eCap), intrinsic nerve activity, and/or electrophysiology (EP).

An eCap is synchronous firing of a population of neurons which occurs in response to the application of a stimulus including, in some cases, an electrical stimulus by a medical device. In some examples, the eCap may be detectable as being a separate event from the stimulus itself, and the Cap may reveal characteristics of the effect of the stimulus on the nerve tissue.

In some examples, expandable substrate 120 may be a stent substrate. In some examples, expandable substrate 120 may include a plurality of fingers 150 protruding from the expandable substrate 120. The plurality of fingers 150 may be positioned at locations circumferentially around the expandable substrate 120 when the expandable substrate 120 is expanded. In some examples, the plurality of fingers 150 may be positioned on a 360° circumferential region of the expandable substrate 120. In some examples, the plurality of fingers 150 may be positioned on less than a 360° circumferential region of the expandable substrate 120. In some examples, the plurality of fingers 150 may be positioned on a particular circumferential quadrant of the expandable substrate 120. In some examples, the plurality of fingers 150 may be positioned on a particular circumferential hemisphere of the expandable substrate 120. In some examples, having the plurality of fingers 150 being positioned in a particular circumferential region, such as a circumferential quadrant or a circumferential hemisphere of the expandable substrate 120 may help identify the circumferential location of the trial therapy delivery location that the stimulation nerve tissue stimulation trial therapy satisfies a target nerve tissue activation threshold.

In some examples, such as shown in FIG. 2C, one or more of the plurality of electrodes 140 may be mounted on a respective finger 150 of the plurality of fingers. In some examples, one of more of the electrodes 140 may comprise a lead wire 130 that extends from the respective electrode 140 and electrically couples to processing circuitry, such as processing circuitry 90 of MD 28. In some examples, such as shown in FIG. 2D, one or more first electrodes 140A comprises a first lead wire 130A. In some examples, such as shown in FIG. 2D, one or more second electrodes 140B comprises a second lead wire 130B. In some examples, each of the one or more first electrodes 140A may comprise a respective first lead wire 130A and each of the one or more second electrodes 140B may comprises a respective second lead wire 130B. In some examples, first lead wire(s) 130A and second lead wire(s) 130B may collectively be referred to as lead wire(s) 130. In some examples, lead wires 130 may comprise one or more of titanium (Ti), tantalum (Ta), tin (Sn), Molybdenum (Mo), Niobium (Nb), Zirconium (Zr), and/or alloys of Ti, Ta, Sn, Mo, Nb, and/or Zr. In some examples, lead wire(s) 130 may be Ta core Ti-15Molybdenum (Mo) or TiTaSn (Ti—52Ta—5Sn) wire. In some examples, lead wires 130 may be insulated such as being coated by an insulating material, such as SI polyimide or PTFE. In some examples, insulated lead wire(s) 130 may form multi-filar coils as part of a lead structure. In some examples, a jacket (tubing) may cover a group of lead wire(s) 130. In some examples, the jacket (tubing) may be thermoplastic polyurethane (TPU), a variation of TPU, or any other suitable elastomer. In some examples, each lead wire 130 may coated by an insulating material. In some examples, one or more lead wire(s) 130 may be an insulated conductor wire formed into coil form and assembled as a lead to connect to an electrical stimulator. In some examples, electrodes 140 may comprise electrode material such as platinum (Pt), PtIridium (Ir) and/or TiTaSn. In some examples, electrodes 140 may be coated with high surface area coating such as one or more of TiN and/or Iridium oxide (IrOx).

In some examples, each of the electrodes 140 may include a communication channel, such as a respective lead wire 130 of the lead wire(s) 130, that are independent of the communication channels (e.g., other lead wire(s) 130) of the other electrodes 140. In some examples, each electrode 140 having a particular insulated lead wire 130 for itself may help electrically isolate each electrode from the other electrodes 140, lead wires 130, and/or expandable substrate 120, which may help enable determination of particular nerve tissue stimulation trial therapy delivered by each particular electrode of the one or more first electrodes 140A and/or enable determination of sensed activation signals by each particular electrode of the one or more second electrodes 140B. In some examples, each of the electrodes 140 may be electrically independent of the other electrodes 140. In some examples, insulating material may be thermoplastic polyurethane (TPU) or other suitable insulating material. In some examples, some of electrodes 140 may be insulated from the expandable substrate 120 with an insulating material covering at least some of the expandable substrate 120 and some of the electrodes 140 may be electrically independent of the other electrodes 140. In some examples, lead wire(s) 130 and/or expandable substrate 120 being insulated may help electrically isolate a particular electrode 140 from the other electrodes 140 which may help a particular electrode 140 be electrically independent from the other electrodes 140. In some examples, one or more of the electrodes 140 may be electrically independent of the other electrodes 140. In some examples, one or more electrodes 140 may be coated with a coating, such as a Titanium Nitride (TiN) alloy, to increase charge density.

In some examples, first lead wires 130A and second lead wires 130B may be configured to be connectable/disconnectable with respect to processing circuitry 90 and connectable to a second processing circuitry, such as processing circuitry of second device 16 or processing circuitry of a chronically implanted stimulation device. In some examples, the second processing circuitry may be positioned in an implantable medical device. For example, in response to a determination that the nerve tissue stimulation trial therapy delivery location satisfies the target nerve tissue activation threshold, first lead wires 130A and/or second lead wires 130B may be disconnected with respect to the processing circuitry 90 and connected to second processing circuitry of an implantable stimulation device configured to deliver the nerve tissue stimulation therapy.

In some examples, endovascular device 112 may be a self-expanding stent or braided structure with one or more electrodes 140 arranged on the outside surface of the structure, such that the electrodes 140 are placed against the inside wall of a respective lumen when the structure expands. In some examples, endovascular device 112 may be a partially deployable stent to temporarily deliver nerve tissue stimulation therapy at the trial therapy delivery location via one or more electrodes 140, such as one or more of electrodes 140A, when the stent is partially deployed. In some examples, endovascular device 112 may be one or more ring like structures with one or more electrodes 140 arranged on the outside surface of the structure such that the electrodes 140 are placed against the inside wall of the respective lumen when the structure expands. In some examples, endovascular device 112 may be a loop structure with one or more electrodes 140 arranged on the outside surface of the structure, such that the electrodes 140 are placed against the inside wall of the respective lumen when the structure expands. In some examples, endovascular device 112 may be a helix structure with one or more electrodes 140 arranged on the outside surface of the structure, such that the electrodes 140 are placed against the inside wall of the respective lumen when the structure expands. In some examples, endovascular device 112 may be resheathable, which may enable removal and/or repositioning while minimizing negative tissues impact. In some examples, endovascular device 112 may be coated with an anti-thrombotic coating. In some examples, expandable substrate 120 may be coated with an anti-thrombotic coating. In some examples, expandable substrate 120 may be partially or entirely coated with an electrically insulating polymer, such as TPU. In some examples, one or more electrodes 140 may be partially coated with an electrically insulating polymer, such as TPU. For example, one or more electrodes 140 may be coated with an electrically insulating polymer on an electrode surface exposed to the lumen of the patient.

In some examples, each of the plurality of electrodes 140 are separated from other electrodes of the plurality electrodes 140 by a distance between 0.5 millimeters (mm) to 5 mm as signal attenuation may be a function of distance between the electrodes. In some examples, some of the plurality of electrodes 140 may be separated from other electrodes of the plurality electrodes 140 by a distance less than 0.5 mm or greater than 5 mm.

In some examples, one or more of electrodes 140 may be positioned on expandable substrate 120 so an exposed portion of a respective electrode 140 face a lumen wall when the expandable substrate 120 is expanded. In some examples, each of the electrodes 140 may be positioned on expandable substrate 120 so an exposed portion of each of the respective electrodes 140 face a lumen wall when the expandable substrate 120 is expanded.

In some examples, as shown in FIG. 2A, endovascular device 112 may include one or more indicators 160. In some examples, the one or more indicators 160 may be attachable/detachable to expandable substrate 120 and may be positioned at various locations on the expandable substrate 120. Endovascular device 112 may be configured to deploy one or more indicators 160 at a particular location. For example, the one or more indicators 160 may be deployed at a particular location to indicate the particular location of the trial therapy delivery location. In some examples, each of the one or more indicators 160 may comprise a radiopaque marker. In some examples, the one or more indicators 160 may be configured to remain at the trial therapy delivery location after removal of the endovascular device 112 from the trial therapy delivery location. In some examples, one or more indicators 160, when located in a lumen of patient 14, may be visible by an imaging device or system. For example, an imaging device or system may identify the particular locations of the one or more indicators 160 and display the identified particular locations of the one or more indicators 160 to help guide an implantable therapy delivery device to be implanted at a location, relative to the displayed particular locations, to deliver nerve tissue stimulation therapy at the trial therapy delivery location, which may help ensure nerve tissue stimulation therapy is delivered at a location of the patient to generate desired patient responsiveness. Guiding an implantable therapy delivery device to be implanted at a particular location that coincides with a successful trial therapy delivery location results in improved patient outcomes and less future invasive procedures to adjust nerve tissue stimulation. This may improve efficiency of nerve tissue stimulation treatment which may reduce overall costs of nerve tissue stimulation treatment. In some examples, such as when endovascular device 112 is removed after determining the nerve tissue stimulation trial therapy delivery location satisfies the target nerve tissue activation threshold and one or more indicators 160 are deployed in the lumen of the patient to identify the particular location to deliver nerve tissue stimulation therapy, an imaging device or system may identify the particular locations of the deployed one or more indicators 160 to help guide an implantable therapy delivery device to be implanted at a location, relative to the deployed one or more indicators, to deliver nerve tissue stimulation therapy at the trial therapy delivery location that satisfies the target nerve tissue activation threshold, which may help ensure nerve tissue stimulation therapy is delivered at a location of the patient that will deliver desired patient responsiveness and results in improved patient outcomes and less future invasive procedures to adjust nerve tissue stimulation. This may improve efficiency of nerve tissue stimulation treatment which may reduce overall costs of nerve tissue stimulation treatment.

In some examples, endovascular device 112 may include a stent that is configured to carry the one or more deployable indicators 160. In some examples, a delivery device may be configured to deliver the endovascular device 112 to a trial therapy delivery location. The one or more indicators 160 may comprise one or more axial indicators and/or one or more rotational indicators that are configured to indicate a position of the endovascular device 112 with respect to the delivery device. In some examples, the one or more indicators 160 may indicate a direction of the one or more electrodes 140. For example, the one or more indicators 160 may comprise a tungsten indicator.

In some examples, an occlusion balloon may be positioned on a proximal end of a delivery device, such as a delivery sheet, to deliver the endovascular device 112 to a trial therapy delivery location. In some examples, an occlusion balloon may be configured to expand to expand a vessel to a particular dimension, such as a maximum expanded dimension, and the delivery device is configured to deliver the endovascular device 112 to the position in the vessel that is expanded by occlusion balloon. In some examples, using an occlusion balloon on a proximal end of a delivery device to help deliver the endovascular device 112 to a trial therapy delivery location may help improve a sufficiency of size endovascular device 112 at the trial therapy location and may help prevent migration of endovascular device 112 after it is positioned at a trial therapy delivery location.

In some examples, endovascular device 112 may also be configured to deliver nerve tissue stimulation therapy at the trial therapy delivery location via one or more electrodes 140, such as one or more of electrodes 140A. For example, endovascular device 112 may be configured to be implanted at the trial therapy delivery location to deliver nerve tissue stimulation therapy, such as in response to a determination that the activation of the target nerve tissue at the trial therapy delivery location satisfies the target nerve tissue activation threshold.

In some examples, endovascular device 112 may be configured to be positioned at or near a brachiocephalic lumen, such as the vein, of the patient to deliver nerve tissue stimulation therapy that stimulates the vagus nerve while reducing laryngeal muscle response. In some examples, endovascular device 112 may be configured to be positioned in a lumen via subclavian access to be positioned at a particular location to deliver nerve tissue stimulation therapy to stimulate a vagus nerve without requiring tunneling to the particular position.

Neurostimulation trial system 2 may deliver nerve tissue stimulation trial therapy, such as stimulation signal(s), to patient 14 by generating and delivering a programmable electrical stimulation signal (e.g., in the form of electrical pulses or an electrical waveform) to target nerve tissue at a trial therapy delivery location near where one or more electrodes 140A are disposed.

FIG. 3 is a block diagram illustrating an example configuration of MD 28. As illustrated in FIG. 3, MD 28 may include one or more of processing circuitry 90, stimulation circuitry 91, memory 92, telemetry circuitry 96, power source 97, sensing circuitry 98, or sensor(s) 99. In some examples, sensor(s) 99 may be instead of or in addition to second device 16. In some examples, a combination of one or more of processing circuitry 90, stimulation circuitry 91, telemetry circuitry 96, and sensing circuitry 98 in MD 28 may generally be referred to as circuitry. Memory 92 may store program instructions that, when executed by processing circuitry 90, cause processing circuitry 90 to provide the functionality ascribed to MD 28 throughout this disclosure. In general, MD 28 may include any suitable arrangement of hardware, alone or in combination with software and/or firmware, to perform the techniques attributed to MD 28, and processing circuitry 90, stimulation circuitry, and telemetry circuitry 96 of MD 28. In various examples, MD 28 may include one or more processors, such as one or more microprocessors, DSPs, ASICs, FPGAs, or any other equivalent integrated or discrete logic circuitry, as well as any combinations of such components. MD 28 also, in various examples, may include a memory 92, such as RAM, ROM, PROM, EPROM, EEPROM, flash memory, a hard disk, a CD-ROM, comprising executable instructions for causing the one or more processors to perform the actions attributed to them. Moreover, although processing circuitry 90 and telemetry circuitry 96 are described as separate circuitry, in some examples, processing circuitry 90 and telemetry circuitry 96 are functionally integrated. In some examples, processing circuitry 90 and telemetry circuitry 96 correspond to individual hardware units, such as microprocessors, ASICs, DSPs, FPGAs, or other hardware units. In other examples, any of processing circuitry 90 and telemetry circuitry 96 may correspond to multiple individual hardware units, such as microprocessors, ASICs, DSPs, FPGAs, or other hardware units.

In some examples, memory 92 may further include program information, e.g., stimulation programs defining the neurostimulation. Generally, stimulation circuitry 91 may generate and deliver electrical stimulation under the control of processing circuitry 90. In some examples, processing circuitry 90 controls stimulation circuitry 91 by accessing memory 92 to selectively access and load at least one of the stimulation programs to stimulation circuitry 91. For example, in operation, processing circuitry 90 may access memory 92 to load a stimulation program to stimulation circuitry 91. In other examples, stimulation circuitry 91 may access memory 92 and load one of the stimulation programs. In some examples, the electrical stimulation signal(s) generated and delivered by stimulation circuitry 91 may have a frequency of 1-10 kHz. In some examples, the electrical stimulation signal(s) generated and delivered by stimulation circuitry 91 may have a frequency of 1-250 Hz. In some examples, the electrical stimulation signal(s) generated and delivered by stimulation circuitry 91 may have a frequency of 1 Hz-10 kHz. In some examples, the electrical stimulation signal(s) generated and delivered by stimulation circuitry 91 may have an amplitude of 0.1-25 mA. In some examples, the electrical stimulation signal(s) generated and delivered by stimulation circuitry 91 may have a pulse width of 10-500 μs.

In some examples, stimulation programs may include stimulation programs that are configured to facilitate different effects. For example, stimulation circuitry may use different stimulation programs to generate different electrical stimulation signals to cause different effects. Stimulation circuitry 91 may deliver stimulation signal(s) to patient 14 for an extended period of time, such as seconds, minutes, hours, days, or until patient 14 or a clinician manually stops or changes the stimulation.

Stimulation circuitry 91 may deliver stimulation signal(s) according to stimulation parameters. Stimulation circuitry 91 may be electrically coupled to some or all of electrodes 140 on endovascular device 112. In some examples, stimulation circuitry 91 may deliver stimulation signal(s) in the form of electrical pulses. In such examples, relevant stimulation parameters may include a voltage amplitude, a current amplitude, a pulse rate, a pulse width, a duty cycle, a duty cycle of the stimulation ON/OFF periods, or the combination of electrodes 140A that stimulation circuitry 91 uses to deliver stimulation signal(s). In other examples, stimulation circuitry 91 may deliver stimulation signal(s) in the form of continuous waveforms. In such examples, relevant stimulation parameters may include a voltage or current amplitude, a frequency, a shape of the stimulation signal, a duty cycle of the stimulation signal, or the combination of electrodes 140A stimulation circuitry 91 uses to deliver the stimulation signal(s).

Sensing circuitry 98 may be electrically coupled to some or all of electrodes 140, on endovascular device 112, particularly electrodes 140B. Sensing circuitry 98 may be coupled to some or all of sensor(s) 99. While FIG. 3 shows sensor(s) 99 as a part of MD 28, in some examples, one or more sensor(s) 99 may be positioned external to MD 28 but are communicatively coupled to MD 28 via the telemetry circuitry 96. Sensing circuitry 98 is configured to obtain signals sensed via one or more combinations of electrodes 140B and/or sensor(s) 99 and process the obtained signals. Sensing circuitry 98 may include one or more filters. In some examples, sensing circuitry 98 may be implemented in the processing circuitry 90 of MD 28. The components of sensing circuitry 98 may be analog components, digital components or a combination thereof. Sensing circuitry 98 may, for example, include one or more sense amplifiers, filters, rectifiers, threshold detectors, analog-to-digital converters (ADCs) or the like. Sensing circuitry 98 may convert the sensed signals to digital form and provide the digital signals to processing circuitry 90 for processing or analysis. For example, sensing circuitry 98 may amplify signals from the sensing electrodes and convert the amplified signals to multi-bit digital signals by an ADC.

Telemetry circuitry 96 supports wireless or wired communication between MD 28, computing device 12, second device 16 and/or any other device. Telemetry circuitry 96 may also be configured to communicate with another computing device via wireless communication techniques, or direct communication through a wired connection. In some examples, telemetry circuitry 96 may provide wireless communication via an RF, proximal inductive medium, or Tissue Conductance Communication (TCC). In some examples, telemetry circuitry 96 may include an antenna, which may take on a variety of forms, such as an internal or external antenna. In some examples, telemetry circuitry 96 may provide communication according to standards or protocols, such as 3G, 4G, 5G, WiFi (e.g., 802.11 or 802.15 ZigBee), Bluetooth®, or Bluetooth® Low Energy (BLE).

Examples of local wireless communication techniques that may be employed to facilitate communication between MD 28 and another computing device include RF communication according to the 802.11 or Bluetooth® specification sets, infrared communication, e.g., according to the IrDA standard, or other standard or proprietary telemetry protocols. In this manner, other external devices may be capable of communicating with MD 28 without needing to establish a secure wireless connection.

Power source 98 delivers operating power to the components of MD 28. Power source 98 may include a battery and a power generation circuit to produce the operating power. In some examples, the battery may be rechargeable to allow extended operation.

In some examples, endovascular device 112 may include an expandable substrate 120 configured to be delivered endovascularly to a trial therapy delivery location in patient 14. Endovascular device 112 may include one or more first electrodes 140A positioned on the expandable substrate 120 and configured to deliver nerve tissue stimulation trial therapy, such as delivering stimulation signal(s), to a target nerve tissue of patient 14. Endovascular device 112 may include one or more second electrodes 140B positioned on the expandable substrate and configured to sense one or more activation signals of the target nerve tissue. In some examples, the one or more electrodes 140A may be configured to deliver nerve tissue stimulation trial therapy, for example one or more stimulation pulses, to a target nerve tissue via one or more first electrodes 140A. In some examples, the one or more second electrodes 140B may sense one or more activation signals of the target nerve tissue in response to the delivery of the stimulation trial therapy to the target nerve tissue. In some examples, the one or more electrodes 140A may be configured to deliver nerve tissue stimulation therapy to the target nerve tissue of the patient 14. In some examples, processing circuitry 90 may cause stimulation circuitry 91 to send one or more stimulation signals to deliver nerve tissue stimulation trial therapy to a target nerve tissue.

In some examples, neurostimulation trial system 2 may include processing circuitry, such as one or more of processing circuitry 90, processing circuitry 13, and/or processing circuitry of second device 16. While references to processing circuitry may refer to processing circuitry 90 of MD 28, any combination of processing circuitry 90, processing circuitry 13, and/or processing circuitry of second device 16 may be used in place of processing circuitry 90.

In some examples, processing circuitry 90 may receive the one of more activation signals sensed by the one or more second electrodes 140B. Processing circuitry 90 may then determine whether activation of the target nerve tissue at the trial therapy delivery location satisfies a target nerve tissue activation threshold based on the one or more sensed activation signals. For example, processing circuitry 90 may determine an amount of activation of the target nerve tissue based on the one or more sensed activation signals and determine whether the amount of activation satisfies the target never tissue activation threshold based on a comparison of the determined amount of activation and the target nerve tissue activation threshold.

In some examples, the sensed activation signals may include one or more of evoked potential data or eCap data. In some examples, the sensed activation signals may include one or more eCap signals. An eCap signal is synchronous firing of a population of neurons which occurs in response to the application of a stimulus including, in some cases, an electrical stimulus by a medical device. In some examples, the eCap signal may be detectable as being a separate event from the stimulus itself, and the eCap signal may reveal characteristics of the effect of the stimulus on the nerve tissue. An eCap signal may refer to a measure of the nerve tissue's response to stimulation, such as nerve tissue stimulation trial therapy. In some examples, eCaps may be a measure of neural recruitment because each eCap signal represents the superposition of electrical potentials generated from a population of axons firing in response to an electrical stimulus (e.g., nerve tissue stimulation trial therapy). Differences in a characteristic (e.g., an amplitude of a portion of the signal or area under the curve of the signal) of eCap signals may occur as a function of how many axons have been activated by the delivered nerve tissue stimulation trial therapy.

In some examples, eCap data may indicate characteristics of activation of particular neural fibers in the nerve tissue. For example, eCap data may indicate whether B-fibers, which may be targeted for heart disease conditions, were activated or C-fibers, which may be targeted for epilepsy, were activated. In some examples, processing circuitry 90 may determine what type of neural fibers in the nerve tissue are activated based on the sensed activation signals to determine whether the target nerve tissue at the trial therapy delivery location is activated.

For example, in response to a stimulation, such as nerve tissue stimulation trial therapy, nerve tissue generates an eCap signal, and the parameters of the eCap signal, such as an amplitude value, may be a function of how much the nerve tissue responded to the stimulation, such as the nerve tissue stimulation trial therapy. In some examples, each electrical stimulation signal in nerve tissue stimulation trial therapy may elicit an eCap signal that is sensed by electrodes 140B. Processing circuitry 90 may receive, via an electrical signal sensed by electrodes 140B, information indicative of an eCap signal (e.g., a numerical value indicating a characteristic of the eCap signal in electrical units such as voltage or power) produced in response to the nerve tissue stimulation trial therapy.

In response to determining that the activation of the target nerve tissue at the trial therapy delivery location satisfies the target nerve tissue activation threshold based on the one or more sensed activation signals, processing circuitry 90 may generate a first indication the trial therapy delivery location of at least one of the first electrodes is acceptable to deliver nerve tissue stimulation therapy. For example, in responses to delivery of the nerve tissue stimulation trial therapy at a trial therapy delivery location, one or more second electrodes 140B may sense an eCap and processing circuitry 90 may compare the sensed eCap to an eCap activation threshold. In response to the sensed eCap satisfying the eCap activation threshold, processing circuitry 90 may generate an indication that the trial therapy delivery location is acceptable to deliver nerve tissue stimulation therapy.

In some examples, the sensed activation signals may include one or more of intrinsic nerve activity signals or intrinsic nerve activity data. In some examples, an intrinsic nerve activity signal is firing of a population of neurons of an intrinsic nerve without providing activation signals to stimulate the nerve immediately beforehand (e.g. within 1 second). In some examples, electrodes 140B may sense intrinsic nerve activity signals. In some examples, electrodes 140B may sense intrinsic nerve activity signals during a period of time after nerve tissue stimulation trial therapy is applied by electrodes 140A. For example, electrodes 140B may sense intrinsic nerve activity signals up to 5 minutes after nerve tissue stimulation trial therapy is applied by electrodes 140A. In some examples, electrodes 140B may sense intrinsic nerve activity signals at a lower sampling rate than eCaps. In some examples, electrodes 140B may be configured to sense intrinsic nerve activity signals between 250 Hz and 4 kHz. In some examples, electrodes 140B may be configured to sense eCaps up to 25 kHz. In some examples, electrodes 140B may be configured to sense eCaps up to 50 kHz. In some examples, sensing intrinsic nerve activity signals at a lower sample rate may reduce power usage.

In some examples, endovascular device 112 may be configured to deliver nerve tissue stimulation therapy via electrodes 140A for a stimulation period of time, such as between 30 seconds and 60 seconds. In some examples, in response to the delivery of nerve tissue stimulation therapy via electrodes 140A, electrodes 140B may sense intrinsic nerve activity signals for a sensing period of time, such as between 3 to 5 minutes after the nerve tissue stimulation therapy is delivered via electrodes 140A. In some examples, electrodes 140B may sense intrinsic nerve activity signals while endovascular device 112 is not delivering nerve tissue stimulation therapy via electrodes 140A.

In some examples, processing circuitry 90 may compare the sensed intrinsic nerve activity signals to a patient intrinsic nerve activity baseline and determine whether the sensed intrinsic nerve activity signals satisfy a patient intrinsic nerve activity baseline threshold. In some examples, in response to a determination that the sensed intrinsic nerve activity signals satisfy a patient intrinsic nerve activity baseline threshold, processing circuitry 90 may cause electrodes 140A to deliver nerve tissue stimulation therapy.

In some examples, in response to processing circuitry 90 determining that the activation of the target nerve tissue at the trial therapy delivery location satisfies the target nerve tissue activation threshold based on the one or more sensed activation signals, processing circuitry 90 may be configured to generate an output indicating the endovascular device 112 be implanted at a location in the patient to deliver nerve tissue stimulation therapy at the trial therapy delivery location via one or more electrodes 140A.

In some examples, one or more second electrodes 140B sensing activation signals may include sensing electrical signals between different electrodes 140B, such as electrodes 140B at different axial positions and/or at different circumferential positions. In some examples, processing circuitry 90 may determine electrical signals between different electrodes of the second electrodes 140B based on the sensed activation signals for each particular electrode 140B of the second electrodes 140B.

In some examples, processing circuitry 90 may generate spectral power

characteristics for one or more frequencies for the detected electrical signals between different electrodes of the second electrodes 140B. Processing circuitry 90 may then determine one or more electrodes 140A combinations of the first electrodes 140A associated with spectral power characteristics indicative of strongest and/or most desirable sensed activation signals, such as eCaps. In some examples, processing circuitry 90 may determine an axial and/or circumferential position of the one or more electrodes 140A associated with the spectral power characteristics indicative of strongest and/or most desirable sensed activation signals. In some examples, higher amplitudes of the spectral power for frequencies indicative of eCaps may indicate that those electrode combinations are close to the originating source of the eCaps.

In some examples, sensing activation signals, such as determining electrical signals between different electrodes 140B, such as electrodes 140B at different axial positions and/or at different circumferential positions, may provide valuable information about where certain electrical signals, such as eCaps, are originating from along the length of nerve tissue. In this manner, processing circuitry 90 may determine the region closest to where the nerve tissue is located and determine that electrodes 140A should be used to deliver nerve tissue stimulation therapy based, at least in part, on the sensed electrical signals between different electrodes 140B.

In some examples, an expandable substrate 120 having both the one or more first electrodes 140A configured to deliver nerve tissue stimulation trial therapy to a target nerve tissue of the patient and the one or more second electrodes 140B configured to sense one or more activation signals of the target nerve tissue positioned on the expandable substrate 120 enables the stimulation and sensing by the system 2 to occur in the same lumen of the patient. This may lead to more accurate determinations of sensed activation signals and/or more accurate determinations of whether activation of the target nerve tissue at the trial therapy delivery location satisfies a target nerve tissue activation threshold based on the one or more sensed activation signals, which may lead to more accurate determinations of positions of electrodes to deliver nerve tissue stimulation therapy that will generate patient responsiveness.

In some examples, in response to determining that the activation of the target nerve tissue at the trial therapy delivery location satisfies the target nerve tissue activation threshold based on the one or more sensed activation signals, processing circuitry 90 may be configured to generate an output indicating endovascular device 112 be implanted at a position in the patient, such as a particular location and/or orientation in a particular lumen, to deliver nerve tissue stimulation therapy at the trial therapy delivery location. In some examples, the position in the patient for device 112 to be implanted is preferably the location and/or orientation in the particular lumen from which it was determined that the activation of the target nerve tissue at the trial therapy delivery location satisfies the target nerve tissue activation threshold.

In some examples, in response to generating the first indication that the trial therapy delivery location of the one or more first electrodes 140 is acceptable to deliver nerve tissue stimulation therapy, processing circuitry 90 may be configured to determine an amount of nerve tissue stimulation therapy to deliver, via the one or more first electrodes 140A, to the target nerve tissue based on one or more of the one or more sensed physiological parameters. In some examples, processing circuitry 90 may be configured to initiate or discontinue delivery of nerve tissue delivery stimulation therapy, via the one or more first electrodes 140A, to the target nerve tissue based on the one or more sensed physiological parameters.

In some examples, processing circuitry 90 may be configured to receive, from a second device, such as second device 16, separate from endovascular device 112, data indicating the one or more physiological parameters of the patient. In some examples, processing circuitry 90 may be configured to determine the target nerve tissue activation threshold based, at least in part, on the received data indicating the one or more physiological parameters of the patient 14. In some examples, data indicating the one or more physiological parameters of the patient 14 may be from various locations in the patient 14, such as epidural, subdural, and/or deep brain tissue. In some examples, data indicating the one or more physiological parameters of the patient 14 may be obtained using various sensing modalities, such as endovascular or parenchymal. In some examples, the received data may indicate one or more of an EEG, ECG, respiration, blood pressure, activity level, EMG, evoked potential, eCap, intrinsic nerve activity, or EP of patient 14. In some examples, the received data may indicate the trial therapy delivery location. In some examples, processing circuitry 90 may be configured to determine a status of a medical event of the patient based, at least in part, on the received data indicating the one or more physiological parameters. In some examples, second device 16 may comprise needle electrodes configured to sense an EMG signal.

In some examples, in response to determining that the activation of the target nerve tissue at the therapy trial delivery location satisfies the target nerve tissue activation threshold based on the one or more sensed activation signals, processing circuitry 90 may be configured to generate an output indicating endovascular device 112 be removed and an implantable therapy delivery device be implanted at a position in the patient to deliver nerve tissue stimulation therapy at the trial therapy delivery location.

In response to a determination that the activation of the target nerve tissue at the trial therapy delivery location does not satisfy the target nerve tissue activation threshold based on the one or more sensed activation signals, processing circuitry 90 may generate a second indication that the trial therapy delivery location of the first electrodes 140A is not acceptable. In some examples, the second indication may include an indication to adjust one or more trial therapy delivery parameters. In some examples, if the activation of the target nerve tissue at the trial therapy delivery location does not satisfy the target nerve tissue activation threshold it may be an indication that that particular location of trial therapy delivery may not be responsive enough to nerve tissue stimulation therapy to achieve desirable patient responsiveness. Accordingly, in some examples, the second indication may include an indication to attempt an additional trial therapy at a second trial therapy delivery location that is different than the initial trial therapy delivery location.

In some examples, circuitry of MD 28 may be configured to adjust the delivery of electrical energy, such as a level of electrical energy, a duration of delivery of electrical energy, or other variables of electrical energy delivery that may be adjusted, to one or more first electrodes 140A to deliver nerve tissue stimulation trial therapy and/or nerve tissue stimulation therapy.

In some examples, the trial therapy delivery parameters may include one or more of amplitude of the stimulation signal(s) of the nerve tissue stimulation trial therapy, pulse width of the stimulation signal(s) of the nerve tissue stimulation trial therapy, frequency of the stimulation signal(s) of the nerve tissue stimulation trial therapy, polarity of the stimulation signal(s) (e.g., monopolar, bipolar, tri-polar or multi-polar), electrode type of the one or more first electrodes 140A, which electrodes of a set of possible first electrodes 140A are used to deliver the nerve tissue stimulation therapy, or location of the one or more first electrodes 140A used to deliver the therapy in a lumen of patient 14. In some examples, the location may include one or more of a longitudinal location on the longitudinal axis with respect to the lumen the endovascular device 112 is inside or a circumferential location with respect to the lumen the endovascular device 112 is inside. In some examples, endovascular device 112 may be partially deployable to temporarily deliver nerve tissue stimulation therapy when the stent is partially deployed. In some examples, endovascular device 112 being partially deployed may include a radius of the endovascular device 112 not being expanded to its full potential radius. In some examples, endovascular device 112 may be partially or completely resheathable. For example, endovascular device 112 may be partially or completely resheathed and repositioned at a different location to attempt to locate a desired therapy location. In some examples, endovascular device 112 may then be partially deployed at the repositioned different location to deliver nerve tissue stimulation therapy when the stent is partially deployed.

In some examples, in response to determining that the activation of the target nerve tissue at the trial therapy delivery location satisfies the target nerve tissue activation threshold based on the one or more sensed activation signals, endovascular device 112 may be configured to provide one or more indicator(s) 160 of the therapy delivery location. In some examples, in response to determining that the activation of the target nerve tissue at the trial therapy delivery location satisfies the target nerve tissue activation threshold based on the one or more sensed activation signals, processing circuitry 90 may generate a signal to deploy the one or more indicator(s) 160 at the therapy delivery location. In some examples, processing circuitry 90 may receive an indication, such as an input from a clinician, to deploy the one or more indicator(s) 160 of the therapy delivery location.

In some examples, one or more second electrodes 140B may be configured to sense one or more physiological parameters of the patient 14. In some examples, the one or more sensed physiological parameters comprises one or more of an EEG, electrocardiogram ECG, respiration, blood pressure, activity level, electromyography EMG, evoked potential, eCap, intrinsic nerve activity, or EP. In some examples, processing circuitry 90 may be configured to initiate nerve tissue stimulation therapy to the target nerve tissue of the patient based on the sensed one or more physiological parameters. In some examples, such as for treating epilepsy, processing circuitry 90 may be configured to initiate nerve tissue stimulation therapy to a vagus nerve of the patient based on sensing ictal tachycardia (an increase of heart rate above a target threshold) via one or more second electrodes 140B. In some examples, processing circuitry 90 may be configured to determine the target nerve tissue activation threshold based, at least in part, on the one or more sensed physiological parameters. In some examples, processing circuitry 90 may be configured to discontinue nerve tissue stimulation therapy to the target nerve tissue of the patient based on the sensed one or more physiological parameters. In some examples, processing circuitry 90 may be configured to discontinue nerve tissue stimulation therapy to a vagus nerve of the patient based on sensing a reduction of heart rate below a threshold via one or more second electrodes 140B.

In some examples, processing circuitry 90 may be configured to determine a status of a medical event of the patient based, at least in part, on the one or more sensed physiological parameters. In some examples, a medical event may be a seizure or an ictal tachycardia. The medical event may also be a variety of other types of medical events, such as, but not limited to, stroke, heart failure (HF), congestive HF, or cardiac arrhythmia.

FIG. 4 is a flow diagram illustrating example techniques according to the present disclosure. One or more first electrodes 140A are positioned on the expandable substrate 120 and configured to deliver the nerve tissue stimulation trial therapy to a target nerve tissue of the patient 14. One or more second electrodes 140B are positioned on expandable substrate 120 and configured to sense one or more activation signals of the target nerve tissue. Processing circuitry 90 may receive the one or more sensed activation signals in response to delivery of the nerve tissue stimulation trial therapy to the target nerve tissue via the one or more first electrodes 140A at the trial therapy delivery location (400). In some examples, processing circuitry 90 may receive the sensed activation signals from electrodes 140B via respective lead wires 130B.

Processing circuitry 90 may then determine whether activation of the target nerve tissue at the trial therapy delivery location satisfies a target nerve tissue activation threshold based on the one or more sensed activation signals (402).

In some examples, in response to processing circuitry 90 determining that the activation of the target nerve tissue at the trial therapy delivery location satisfies the target nerve tissue activation threshold based on the one or more sensed activation signals, processing circuitry 90 may generate an indication that the trial therapy delivery location of at least one of the first electrodes 140A is acceptable to deliver nerve tissue stimulation therapy (404).

In some examples, in response to processing circuitry 90 determining that the activation of the target nerve tissue at the trial therapy delivery location does not satisfy the target nerve tissue activation threshold based on the one or more sensed activation signals, processing circuitry 90 may generate an indication that the trial therapy delivery location of the first electrodes is not acceptable to deliver nerve tissue stimulation therapy (406).

In some examples, in response to processing circuitry 90 determining that the activation of the target nerve tissue at the trial therapy delivery location does not satisfy the target nerve tissue activation threshold based on the one or more sensed activation signals and upon generating an indication the trial therapy delivery location of the first electrodes is not acceptable to deliver nerve tissue stimulation therapy, processing circuitry 90 may generate an indication to adjust trial therapy delivery parameters (408). In some examples, the trial therapy delivery parameters comprise one or more of amplitude, pulse width, frequency, polarity (e.g., monopolar, bipolar, tri-polar, or multi-polar), electrode 140A type, or electrode 140A location. In some examples, the generating of an indication to adjust trial therapy delivery parameters may coincide with the generating an indication the trial therapy delivery location of the first electrodes is not acceptable to deliver nerve tissue stimulation therapy.

The techniques of this disclosure may trial nerve tissue stimulation on patients to test therapy responsiveness and determine adequate and/or optimum placement of electrodes configured to deliver nerve tissue stimulation therapy, which may provide more accurate, more efficient, and/or less invasive implantation of a nerve tissue stimulation device which may lead to improved patient responsiveness from nerve tissue stimulation therapy.

It should be noted that the techniques described herein, may not be limited to treatment or monitoring of a human patient. In alternative examples, the techniques of this disclosure may be applied to non-human patients, e.g., primates, canines, equines, pigs, and felines. These other animals may undergo clinical or research therapies that my benefit from the subject matter of this disclosure.

Various examples are discussed relative to one or more neurostimulation trial devices and systems. It is recognized that the stimulation devices and/or systems may include features and functionality in addition to electrical stimulation. Many of these additional features are expressly discussed herein. A few example features include, but are not limited to, different types of sensing capabilities and different types of wireless communication capabilities. For ease of discussion, the present disclosure does not expressly recite every conceivable combination of the additional features, such as by repeating every feature each time different examples and uses of the stimulation devices and/or systems are discussed.

The techniques of this disclosure may be implemented in a wide variety of computing devices, medical devices, or any combination thereof. Any of the described units, circuitry or components may be implemented together or separately as discrete but interoperable logic devices. Depiction of different features as circuitry or units is intended to highlight different functional aspects and does not necessarily imply that such circuitry or units must be realized by separate hardware or software components. Rather, functionality associated with one or more circuitry or units may be performed by separate hardware or software components, or integrated within common or separate hardware or software components.

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory that is tangible. The computer-readable storage media may be referred to as non-transitory. A server, client computing device, or any other computing device may also contain a more portable removable memory type to enable easy data transfer or offline data analysis.

The techniques described in this disclosure, including those attributed to various circuitry and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated, discrete logic circuitry, or other processing circuitry, as well as any combinations of such components, remote servers, remote client devices, or other devices. The term “processing circuitry” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, circuitry or components may be implemented together or separately as discrete but interoperable logic devices. Depiction of different features as circuitry or units is intended to highlight different functional aspects and does not necessarily imply that such circuitry or units must be realized by separate hardware or software components. Rather, functionality associated with one or more circuitry or units may be performed by separate hardware or software components, or integrated within common or separate hardware or software components. For example, any circuitry described herein may include electrical circuitry configured to perform the features attributed to that particular circuitry, such as fixed function processing circuitry, programmable processing circuitry, or combinations thereof.

In some examples, a computer-readable storage medium comprises non-transitory medium. The term “non-transitory” may indicate that the storage medium is not embodied in a carrier wave or a propagated signal. In certain examples, a non-transitory storage medium may store data that may, over time, change (e.g., in RAM or cache).

This disclosure includes the following non-limiting examples.

Example 1: A system includes processing circuitry; and an endovascular device includes an expandable substrate configured to be delivered endovascularly to a trial therapy delivery location in a patient; one or more first electrodes positioned on the expandable substrate, wherein the one or more first electrodes are configured to deliver nerve tissue stimulation trial therapy to a target nerve tissue of the patient; and one or more second electrodes positioned on the expandable substrate, wherein the one or more second electrodes are configured to sense one or more activation signals of the target nerve tissue, wherein the processing circuitry is configured to: in response to delivery of the nerve tissue stimulation trial therapy to the target nerve tissue via the one or more first electrodes at the trial therapy delivery location, receive, via the one or more second electrodes, the one or more sensed activation signals; determine whether activation of the target nerve tissue at the trial therapy delivery location satisfies a target nerve tissue activation threshold based on the one or more sensed activation signals; in response to determining that the activation of the target nerve tissue at the trial therapy delivery location satisfies the target nerve tissue activation threshold based on the one or more sensed activation signals, generate a first indication that the trial therapy delivery location of at least one of the first electrodes is acceptable to deliver nerve tissue stimulation therapy; and in response to determining that the activation of the target nerve tissue at the trial therapy delivery location does not satisfy the target nerve tissue activation threshold based on the one or more sensed activation signals, generate a second indication that the trial therapy delivery location of the first electrodes is not acceptable to deliver nerve tissue stimulation therapy.

Example 2: The system recited in example 1, wherein to determine that the activation of the target nerve tissue satisfies the target nerve tissue activation threshold, the processing circuitry is configured to: determine an amount of activation of the target nerve tissue based on the one or more sensed activation signals; and determine that the amount of activation of the target nerve tissue satisfies the target nerve tissue activation threshold based on a comparison of the determined amount of activation and the target nerve tissue activation threshold.

Example 3: The system recited in any of examples 1-2, wherein in response to determining that the activation of the target nerve tissue at the trial therapy delivery location satisfies the target nerve tissue activation threshold based on the one or more sensed activation signals, the processing circuitry is configured to generate an output indicating the endovascular device be implanted at a location in the patient to deliver nerve tissue stimulation therapy at the trial therapy delivery location.

Example 4: The system recited in any of examples 1-2, wherein in response to determining that the activation of the target nerve tissue at the therapy trial delivery location satisfies the target nerve tissue activation threshold based on the one or more sensed activation signals, the processing circuitry is configured to generate an output indicating the endovascular device be removed and an implantable therapy delivery device be implanted at a position in the patient to deliver nerve tissue stimulation therapy at the trial therapy delivery location.

Example 5: The system recited in any of examples 1-4, wherein the endovascular device is further configured to provide an indicator of the trial therapy delivery location.

Example 6: The system recited in example 5, wherein the indicator is configured to remain at the trial therapy delivery location after removal of the endovascular device from the trial therapy delivery location.

Example 7: The system recited in any of examples 5-6, wherein the indicator comprises a radiopaque marker.

Example 8: The system recited in any of examples 5-7, wherein the endovascular device further comprises a stent configured to carry the indicator.

Example 9: The system recited in any of examples 5-8, wherein the indicator is configured to attach to a delivery device configured to deliver the endovascular device to the trial therapy delivery location, and the indictor comprises axial indicators and rotational indicators configured to indicate a position of the endovascular device with respect to the delivery device.

Example 10: The system recited in any of examples 1-9, wherein the one or more second electrodes are configured to sense one or more physiological parameters of the patient.

Example 11: The system recited of example 10, wherein the processing circuitry is further configured to initiate nerve tissue stimulation therapy to the target nerve tissue of the patient based on the sensed one or more physiological parameters.

Example 12: The system recited of example 11, wherein the processing circuitry is further configured to determine the target nerve tissue activation threshold based, at least in part, on the one or more sensed physiological parameters.

Example 13: The system recited in any of examples 10-12, wherein the one or more sensed physiological parameters comprises one or more of an electroencephalogram (EEG), electrocardiogram (ECG), respiration, blood pressure, activity level, electromyography (EMG), evoked potential, evoked compound action potential (eCap), intrinsic nerve activity, or electrophysiology (EP).

Example 14: The system recited of any of examples 10-13, wherein the processing circuitry is further configured to determine a status of a medical event of the patient based, at least in part, on the one or more sensed physiological parameters.

Example 15: The system recited in any of examples 1-14, wherein the processing circuitry is further configured to receive, from a second device separate from the endovascular device, data indicating one or more physiological parameters of the patient.

Example 16: The system recited of example 15, wherein the processing circuitry is further configured to determine the target nerve tissue activation threshold based, at least in part, on the received data indicating one or more physiological parameters of the patient.

Example 17: The system recited in any of examples 15-16, wherein the received data indicating one or more physiological parameters comprises one or more of an electroencephalogram (EEG), electrocardiogram (ECG), respiration, blood pressure, activity level, electromyography (EMG), evoked potential or evoked compound action potential (eCap), intrinsic nerve activity, or electrophysiology (EP).

Example 18: The system recited of any of examples 16-17, wherein the processing circuitry is further configured to determine a status of a medical event of the patient based, at least in part, on the received data indicating one or more physiological parameters.

Example 19: The system recited in any of examples 14 or 18, wherein the medical event is a seizure.

Example 20: The system recited in any of examples 14 or 18, wherein the medical event is an ictal tachycardia.

Example 21: The system recited in any of examples 10-20, wherein, the one or more first electrodes are further configured to deliver nerve tissue stimulation therapy to the target nerve tissue of the patient, and in response to generating the first indication that the trial therapy delivery location of the one or more first electrodes is acceptable to deliver nerve tissue stimulation therapy, the processing circuitry is further configured to determine an amount of nerve tissue stimulation therapy to deliver, via the one or more first electrodes, to the target nerve tissue based on one or more of the one or more sensed physiological parameters.

Example 22: The system recited in any of examples 1-21, wherein each of the one or more first electrodes comprises a first lead wire coupled to the processing circuitry, and each of the one or more second electrodes comprises a second lead wire coupled to the processing circuitry.

Example 23: The system recited in any of examples 1-22, wherein the first lead wires and the second lead wires are configured to be disconnected with respect to the processing circuitry and connected to a second processing circuitry, the second processing circuitry being positioned in an implantable medical device.

Example 24: The system recited in any of examples 1-23, wherein the second indication includes an indication to adjust one or more trial therapy delivery parameters.

Example 25: The system recited in example 24, wherein the trial therapy delivery parameters comprise one or more of amplitude, pulse width, frequency, electrode type, polarity, or electrode location.

Example 26: The system recited in any of examples 1-25, wherein the endovascular device is resheathable.

Example 27: The system recited in any of examples 1-26, wherein the target nerve tissue comprises a vagus nerve of the patient.

Example 28: An endovascular device includes a stent substrate configured to be delivered endovascularly to a trial therapy delivery location in a patient; a plurality of fingers protruding from a plurality of positions on the stent substrate; one or more indicators; and a plurality of electrodes, the plurality of electrodes are configured to perform one or more of: deliver nerve tissue stimulation trial therapy to a target nerve tissue of the patient, and sense one or more activation signals of the target nerve tissue, wherein one of the plurality of electrodes or one of the one or more indicators are mounted on a respective finger of the plurality of fingers.

Example 29: The endovascular device recited in example 28, wherein the plurality of fingers are positioned at locations circumferentially around the stent substrate.

Example 30: The endovascular device recited in example 28, wherein the plurality of fingers are positioned only at locations on a particular circumferential region of the stent substrate.

Example 31: The endovascular device recited in any of examples 28-30, wherein each of the plurality of electrodes are separated from other electrodes of the plurality electrodes by a distance between 0.5 millimeters (mm) to 5 mm.

Example 32: The endovascular device recited in any of examples 28-31, wherein at least one of the plurality of fingers is configured to lock upon one of the plurality of electrodes being mounted on the respective finger.

Example 33: The endovascular device recited in any of examples 28-32, wherein the indicator comprises a radiopaque marker.

Example 34: The endovascular device recited in any of examples 28-33, wherein one or more of the plurality of electrodes are attached to a respective finger and attached to an independent and insulated lead wire.

Example 35: The endovascular device recited in any of examples 28-34, wherein the endovascular device is resheathable.

Example 36: An endovascular device includes a stent substrate including a plurality of struts and configured to be delivered endovascularly to a trial therapy delivery location in a patient; and a plurality of electrodes, the plurality of electrodes are configured to perform one or more of: deliver nerve tissue stimulation trial therapy to a target nerve tissue of the patient, and sense one or more activation signals of the target nerve tissue, wherein one of more of the plurality of electrodes are wrapped around a respective single strut of the plurality of struts and attached to an independent and insulated lead wire.

Example 37: The endovascular device recited in example 36, wherein the endovascular device further comprises one or more indicators attachable to the stent substrate.

Example 38: The endovascular device recited in any of examples 36-37, wherein each of the plurality of electrodes are separated from other electrodes of the plurality electrodes by a distance between 0.5 millimeters (mm) to 5 mm.

Example 39: The endovascular device recited in any of examples 36-38, wherein the endovascular device is resheathable.

Various examples have been described herein. Any combination of the described operations or functions is contemplated. These and other examples are within the scope of the following claims. Based upon the above discussion and illustrations, it is recognized that various modifications and changes may be made to the disclosed examples in a manner that does not require strictly adherence to the examples and applications illustrated and described herein. Such modifications do not depart from the true spirit and scope of various aspects of the disclosure, including aspects set forth in the claims.

What is claimed is:

1. A system comprising:
processing circuitry; and
an endovascular device comprising:
an expandable substrate configured to be delivered endovascularly to a trial therapy delivery location in a patient;
one or more first electrodes positioned on the expandable substrate, wherein the one or more first electrodes are configured to deliver nerve tissue stimulation trial therapy to a target nerve tissue of the patient; and
one or more second electrodes positioned on the expandable substrate, wherein the one or more second electrodes are configured to sense one or more activation signals of the target nerve tissue,
wherein the processing circuitry is configured to:
in response to delivery of the nerve tissue stimulation trial therapy to the target nerve tissue via the one or more first electrodes at the trial therapy delivery location, receive, via the one or more second electrodes, the one or more sensed activation signals;
determine whether activation of the target nerve tissue at the trial therapy delivery location satisfies a target nerve tissue activation threshold based on the one or more sensed activation signals;
in response to determining that the activation of the target nerve tissue at the trial therapy delivery location satisfies the target nerve tissue activation threshold based on the one or more sensed activation signals, generate a first indication that the trial therapy delivery location of at least one of the first electrodes is acceptable to deliver nerve tissue stimulation therapy; and
in response to determining that the activation of the target nerve tissue at the trial therapy delivery location does not satisfy the target nerve tissue activation threshold based on the one or more sensed activation signals, generate a second indication that the trial therapy delivery location of the first electrodes is not acceptable to deliver nerve tissue stimulation therapy.

2. The system recited in claim 1, wherein to determine that the activation of the target nerve tissue satisfies the target nerve tissue activation threshold, the processing circuitry is configured to:
determine an amount of activation of the target nerve tissue based on the one or more sensed activation signals; and
determine that the amount of activation of the target nerve tissue satisfies the target nerve tissue activation threshold based on a comparison of the determined amount of activation and the target nerve tissue activation threshold.

3. The system recited in claim 1, wherein in response to determining that the activation of the target nerve tissue at the trial therapy delivery location satisfies the target nerve tissue activation threshold based on the one or more sensed activation signals, the processing circuitry is configured to generate an output indicating the endovascular device be implanted at a location in the patient to deliver nerve tissue stimulation therapy at the trial therapy delivery location.

4. The system recited in claim 1, wherein in response to determining that the activation of the target nerve tissue at the therapy trial delivery location satisfies the target nerve tissue activation threshold based on the one or more sensed activation signals, the processing circuitry is configured to generate an output indicating the endovascular device be removed and an implantable therapy delivery device be implanted at a position in the patient to deliver nerve tissue stimulation therapy at the trial therapy delivery location.

5. The system recited in claim 1, wherein the endovascular device is further configured to provide an indicator of the trial therapy delivery location.

6. The system recited in claim 5, wherein the indicator is configured to attach to a delivery device configured to deliver the endovascular device to the trial therapy delivery location, and the indictor comprises axial indicators and rotational indicators configured to indicate a position of the endovascular device with respect to the delivery device.

7. The system recited in claim 1, wherein the one or more second electrodes are configured to sense one or more physiological parameters of the patient.

8. The system recited of claim 7, wherein the processing circuitry is further configured to initiate nerve tissue stimulation therapy to the target nerve tissue of the patient based on the sensed one or more physiological parameters.

9. The system recited of claim 8, wherein the processing circuitry is further configured to determine the target nerve tissue activation threshold based, at least in part, on the one or more sensed physiological parameters.

10. The system recited in claim 7, wherein the one or more sensed physiological parameters comprises one or more of an electroencephalogram (EEG), electrocardiogram (ECG), respiration, blood pressure, activity level, electromyography (EMG), evoked potential, evoked compound action potential (eCap), intrinsic nerve activity, or electrophysiology (EP).

11. The system recited of claim 10, wherein the processing circuitry is further configured to determine a status of a medical event of the patient based, at least in part, on the one or more sensed physiological parameters.

12. The system recited in claim 11, wherein the medical event is a seizure or an ictal tachycardia.

13. The system recited in claim 7, wherein, the one or more first electrodes are further configured to deliver nerve tissue stimulation therapy to the target nerve tissue of the patient, and
in response to generating the first indication that the trial therapy delivery location of the one or more first electrodes is acceptable to deliver nerve tissue stimulation therapy, the processing circuitry is further configured to determine an amount of nerve tissue stimulation therapy to deliver, via the one or more first electrodes, to the target nerve tissue based on one or more of the one or more sensed physiological parameters.

14. The system recited in claim 1, wherein the second indication includes an indication to adjust one or more trial therapy delivery parameters.

15. An endovascular device comprising:
a stent substrate configured to be delivered endovascularly to a trial therapy delivery location in a patient;
a plurality of fingers protruding from a plurality of positions on the stent substrate;
one or more indicators; and
a plurality of electrodes, the plurality of electrodes are configured to perform one or more of:
deliver nerve tissue stimulation trial therapy to a target nerve tissue of the patient, and
sense one or more activation signals of the target nerve tissue,
wherein one of the plurality of electrodes or one of the one or more indicators are mounted on a respective finger of the plurality of fingers.

16. The endovascular device in claim 15, wherein the plurality of fingers are positioned at locations circumferentially around the stent substrate.

17. The endovascular device in claim 15, wherein at least one of the plurality of fingers is configured to lock upon one of the plurality of electrodes being mounted on the respective finger.

18. The endovascular device in claim 15, wherein one or more of the plurality of electrodes are attached to a respective finger and attached to an independent and insulated lead wire.

19. An endovascular device comprising:
a stent substrate including a plurality of struts and configured to be delivered endovascularly to a trial therapy delivery location in a patient; and
a plurality of electrodes, the plurality of electrodes are configured to perform one or more of:
deliver nerve tissue stimulation trial therapy to a target nerve tissue of the patient, and
sense one or more activation signals of the target nerve tissue,
wherein one of more of the plurality of electrodes are wrapped around a respective single strut of the plurality of struts and attached to an independent and insulated lead wire.

20. The endovascular device in claim 19, wherein the endovascular device further comprises one or more indicators attachable to the stent substrate.
